(19) United States
(12) Patent Application Publication
(10) Pub. No.: US 2006/0116658 A1
(43) Pub. Date: Jun. 1, 2006

(54) Multi-lumen stoma measuring device and method for using same

(75) Inventors: Donald J. McMichael, South Jordan, UT (US); Mark E. Foster, Sandy, UT (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/000,231

(22) Filed: Nov. 30, 2004

(51) Int. Cl. A61M 31/00

(52) U.S. Cl. 604/509; 604/104; 604/96.01; 604/910

(57) ABSTRACT

A multi-lumen stoma or tract measuring device and method of using the same. The measuring device generally including a multi-lumen shaft, a head secured to one end of the shaft, and a deployable retention member secured to the shaft. The head having an opening in communication with each of the lumens, the deployable retention member being in communication with one of the lumens of the shaft. The multi-lumen device adapted to receive a guidewire therethrough.

Catheterization of a body cavity is frequently performed in medical procedures either to insert substances into or to remove substances from the body. During many of these procedures, it is necessary to keep the catheter in a relatively stable position to perform the desired insertion or removal. With the use of enteral feeding catheters (i.e., catheters which enable the administration of nutritional solutions directly into the stomach or intestines), for example, it is necessary to ensure that the catheter is not accidentally removed from the stomach or intestines. This is true both during the actual administration or removal of fluids, and the time periods in between.

In order to ensure that a catheter is maintained in the proper position, it is common to use a balloon disposed near the distal (patient) end of the catheter shaft. Inflating the balloon causes the balloon to contact the anatomical structure (i.e., a duct or stomach wall) and thereby prevents the catheter from moving out of the proper position. In the case of enteral feeding, a stoma is formed leading into the stomach or intestine. The catheter is positioned to extend through the stoma so as to form a channel into the stomach or intestines through which enteral feeding solutions may be instilled.

FIG. 1 shows a side view of a prior art balloon catheter 10 having a head 14 disposed at a proximal end 15 of the catheter 10. The head 14 contains valves (not shown) which regulate the flow of fluids through the balloon catheter 10. The head 14 also prevents the balloon catheter 10 from completely advancing through the stoma and into the stomach or intestine of the user.

To prevent the catheter 10 from being pulled out of the stomach/intestinal wall, a balloon 18 is disposed along a catheter shaft 26. The catheter 10 is shown having an optional stiff tip 30, which is attached to the catheter shaft 26 at a distal end 17 opposite the head 14. The catheter shaft 26 is typically made of a medical grade silicone. The stiff tip 30, when present, is also frequently formed of a medical grade silicone but is usually configured to be at least as rigid as the catheter shaft 26.

The balloon 18 has a balloon proximal end 20 attached to the catheter shaft 26 by the use of adhesive, thereby forming a proximal cuff 32. Likewise, the balloon distal end 22 is adhesively attached to the catheter shaft 26 and/or stiff tip 30, thereby forming a distal cuff 34.

The balloon 18 is advantageous because it allows the catheter shaft 26 to be inserted into the stoma (not shown) while the balloon 18 is uninflated. Once the catheter shaft 26 is properly positioned in the stoma, a syringe (not shown) is inserted into a side port 36 of the head 14 and a fluid is injected into the balloon 18 through a lumen (not shown in FIG. 1) of the catheter 10 so as to inflate the balloon 18.

While the balloon 18 remains inflated, the catheter 10 stays properly positioned in the stoma. The position of the balloon catheter 10 is maintained in such a manner until removal is desired. If the catheter 10 needs to be removed, the balloon 18 may be deflated so that it will not interfere with withdrawal of the catheter shaft 26 and stiff tip 30.

The type of balloon 18 shown in FIG. 1 is fashioned around the perimeter of the catheter shaft 26 such that when it is deflated it reduces or contracts about the shaft 26 but is still clearly larger than overall diameter of the catheter.

Attachment of the balloon 18 to the catheter shaft 26 is frequently accomplished by gluing the balloon proximal end 20 and the balloon distal end 22 to corresponding positions on the external surface of the catheter shaft 26 so as to form a proximal cuff 32 and a distal cuff 34, respectively. Such cuffs 32 and 34 are longitudinal sections of the balloon 18 whose inside diameters correspond to the outside diameter of the shaft 26 at their respective points of attachment to the catheter 10 and have a distance between them which is about the length of the uninflated balloon 18. The cuffs 32 and 34 must be of sufficient length to provide a tight and durable seal between the balloon 18 and the catheter shaft 26.

FIG. 2 shows a side view of another prior art balloon catheter 110. The catheter 110 is generally similar to catheter 10 (FIG. 1) except that the head 114 (FIG. 2) of catheter 110 is a large or non-low profile head and is adapted to extend well beyond the patient's body. While the balloon 18 of catheter 10 may be located at or near the distal end 17 of catheter shaft 26, as shown in FIG. 1, FIG. 2 also shows that balloon 118 may be located more inwardly of the distal end 117 of the catheter 110 (i.e. more proximal to the head 114).

While the prior art balloon configurations shown in FIGS. 1 and 2 work to maintain the balloon catheters 10 and 110, respectively, in the proper position within the patient, those balloon catheters as well as the other known balloon catheters do have disadvantages, especially involving placement. For example, sizing a catheter is important to minimize the trauma to a patient. If a catheter being is too small it may cause undue pressure to be exerted on or unnecessarily constrict the patient's tissue. If a catheter is too big, slippage may occur, and the repeated sliding of the catheter along the stoma or tract may lead to irritation and/or infection. The sizing issues are especially significant with low profile enteral feeding devices as the low profile devices are generally not adjustable for different stoma or tract lengths.

Additional difficulties with prior measuring devices are commonly encountered with or during the placement or replacement of longer enteral feeding devices such as transgastic jejunal devices. Frequently, these longer tubes are placed with the assistance of a guidewire. That is, many enteral catheters such as trans-gastric jejunal tubes need and/or desirably utilize guidewire placement. In these cases a guidewire is often left in place during removal of the old device and the tract or stoma length must be measured with the guidewire in place. The ability to accurately measure a stoma or tract size has been difficult in the past where a guidewire is present. As prior devices did not accommodate the passage of the guidewire therethrough, the insertion of a stoma measuring device required that the guidewire be sandwiched between the tract or stoma and the outside of the stoma measuring device. This can cause difficulty in positioning the stoma measuring device and/or it can cause irritation of the stoma during insertion or removal of the stoma measuring device. In either case, it is common with prior stoma measuring devices for the guidewire or at least the distal end thereof to be moved or dislodged from its desired position thereby further complicating placement of the enteral feeding device.

Accordingly, there is a need and desire for a stoma or tract measuring device which is capable of accommodating the passage of a guidewire therethrough.

### SUMMARY OF THE INVENTION

In response to the difficulties and problems discussed above, an improved tract measuring device has been developed.

One aspect of the present invention is tract measuring device which may be used to determine the length of a stoma or other tract opening within a patient. The tract measuring device is adapted to receive or pass therethrough a guidewire which is placed in the patient to help place a replacement catheter. The device generally includes a head having at least two openings; a shaft extending from the head, the shaft having a first and second lumen disposed in communication with the at least two openings, the first lumen configured for communication with a body cavity; and a sleeve having a proximal end and a distal end, wherein each end of the sleeve is attached to the shaft; wherein an expandable cavity is formed between the sleeve and the shaft and is in communication with the second lumen in the shaft. The measuring device may also include scale indicia along at least a portion of the shaft.

The present invention is also directed to a dual lumen tract measuring device generally including a shaft and a retention member. The shaft has two lumens, a length, and scale indicia along at least a portion of the length, and the retention member is adapted for deployment. The first lumen should extend the length of the shaft, and the second lumen is in communication with the retention member such that the retention member is deployable through communication with the second lumen.

The present invention is also directed to a method of measuring a tract in a patient. The method generally includes the steps of: providing a measuring device such as those discussed immediately above; inserting the distal end of the measuring device into the tract in the patient; deploying or expanding the sleeve of the device; positioning the measuring device such that a proximal edge of the expanded sleeve rests against an inner surface of an inner body cavity within the patient; and determining the distance between the proximal edge of the expanded sleeve and the outer surface of the patient's abdominal wall.

The present invention is also directed to a method of providing a system for determining the size of tract in a patient. The method generally includes the steps of: providing a tract measuring device having at least one measuring means and providing directions for positioning the measuring device relative to the patient so as to allow a clinician to determine the size of the tract utilizing the at least one measuring means. The measuring device may generally include: a head having at least two openings; a shaft extending from the head, the shaft having a first and second lumen disposed in communication with the at least two openings, the first lumen configured for communication with a body cavity; and a sleeve having a proximal end and a distal end, wherein each end of the sleeve is attached to the shaft; wherein an expandable cavity is formed between the sleeve and the shaft and is in communication with the second lumen in the shaft. The measuring device may further include scale indicia along at least a portion of the shaft, and the method may further include the step of utilizing the scale indicia to determine the size of the tract.

The invention will be more fully understood and further features and advantages will become apparent when reference is made to the following detailed description of exemplary aspects of the invention and the accompanying drawings.

### BRIEF DESCRIPTION OF THE DRAWINGS

The purpose and advantages of the present invention will be apparent to those skilled in the art from the following detailed description in conjunction with the appended drawings in which:

FIG. 1 is a view of a prior enteral feeding device;

FIG. 2 is a view of another prior enteral feeding device;

FIG. 3 is a side view of an aspect of a measuring device according to the present invention;

FIG. 4 is a cross-sectional view of the device of FIG. 3 taken along line 3-3′;

FIG. 5 is a cross-sectional view of the device of FIG. 3 having an alternate sleeve attachment;

FIG. 6 is an perspective view of an aspect of the measuring device according to the present invention wherein the measuring device is shown positioned within a patient.

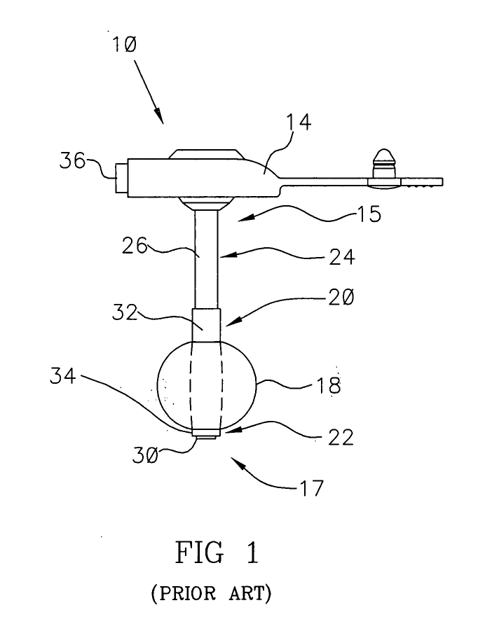

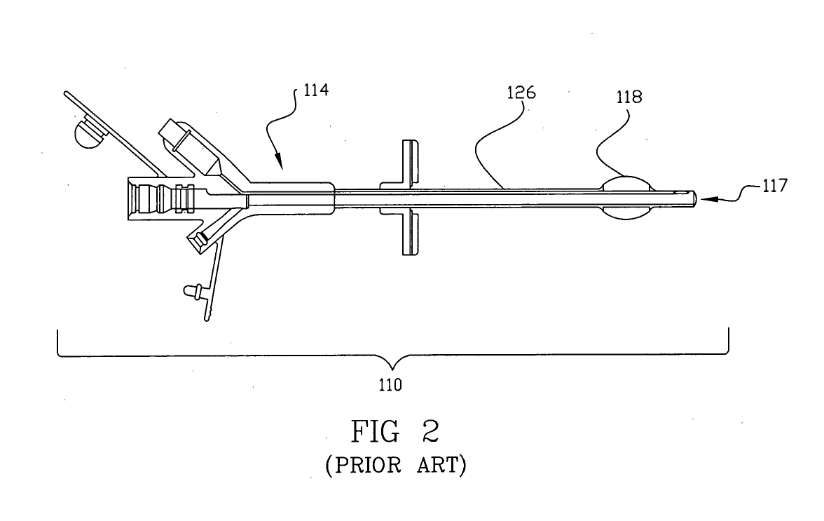

### DETAILED DESCRIPTION OF THE PRESENT INVENTION

Reference now will be made in detail to various embodiments of the invention, one or more examples of which are set forth below. Various elements of the present invention will be given numeral designations and the invention will be discussed so as to enable one skilled in the art to make and use the invention. It should be appreciated that each example is provided by way of explaining the invention, and not as a limitation of the invention. For example, features illustrated or described with respect to one aspect may be used with another aspect to yield still a further aspect. These and other modifications and variations are contemplated to be within the scope and spirit of the invention.

In addition, the invention will be described in the context of its various configurations. It should be appreciated that alternative arrangements of the invention can comprise any combination of such configurations. As such, the use of a desired aspect for ease in understanding and describing the invention shall not, in any manner, limit the scope of the invention.

As used herein, the term “distal” generally refers to the direction of the patient or the end of a device intended to be closest to or inserted the farthest into a patient and the term “proximal” generally refers to the direction of the clinician or the end of a device intended to be furthest from or inserted the least into a patient.

As used herein the term stoma or tract measuring device generally refers to a device intended to be introduced into an opening in a body and to allow for measurement of the length or depth of the opening.

Although the term “sleeve” is generally used throughout, it is also intended to include, but is not limited to, other expandable members such as balloons or the like. As used herein, the term “retention member” is intended to include, but is not limited to, expandable or deployable members of any sort, including, but not limited to, sleeves, moly-bolts, or the like.

It will be appreciated that as used herein the terms expanding and deploying or deviations thereof are intended to overlap in meaning and be used interchangeably herein.

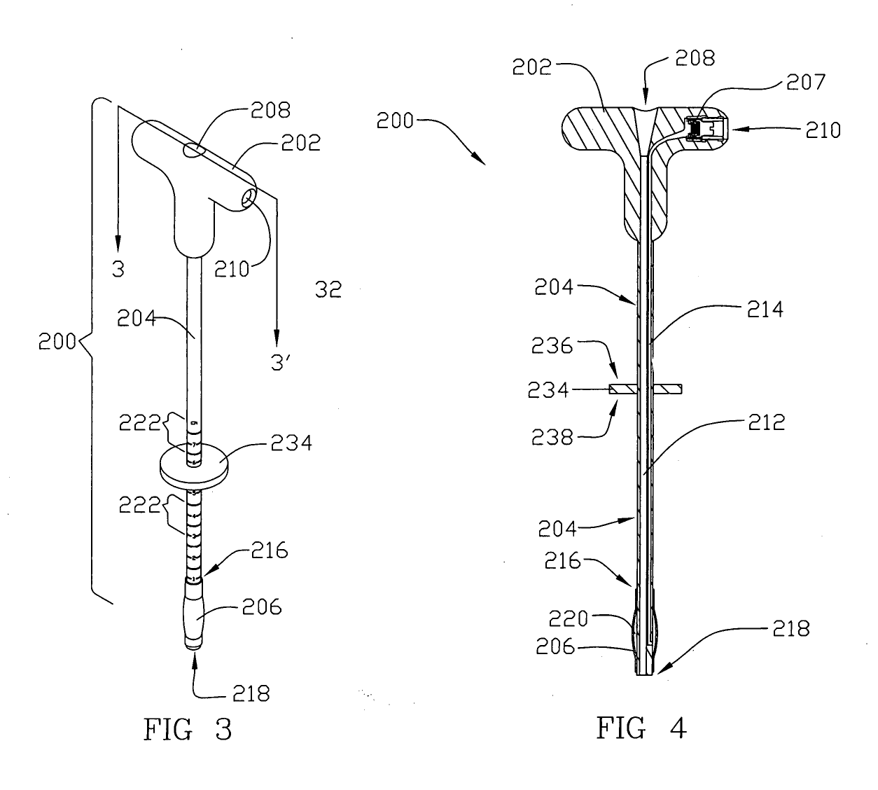

FIG. 3 illustrates a multi-lumen guidewire accessible tract measuring device 200 according to the present invention. The tract measuring device may be used to determine the length of a particular stoma or tract in a patient. Such a device is especially useful in determining the proper size of an enteral feeding device to be placed in the patient through the stoma. As illustrated, the measuring device 200 includes a head 202, a shaft 204 extending from the head 202, and a sleeve 206. The head 202 has at least two openings 208, 210. The shaft 204 is shown in FIG. 4 having a first lumen 212 and second lumen 214 disposed in communication with the two openings 208 and 210, respectively. The first lumen 212 being configured for communication with a body cavity. The sleeve 206 is shown having a proximal end 216 and a distal end 218, wherein each end of the sleeve 206 is attached to the shaft 204. The sleeve 206 is positioned about the shaft 204 such that an expandable cavity 220 is formed between the sleeve 206 and the shaft 204 and is in communication with the second lumen 214 in the shaft 204.

The measuring device may further include scale indicia such as that shown as 222 along at least a portion of the shaft 204. The scale indicia may take any suitable form or color. It will be appreciated that all suitable sizes and scales of markings or indicia are contemplated. At least one aspect of the present invention contemplates measurement markings every 0.25 cm for a total of 6.0 cm. It is contemplated that the markings may be created in or on the shaft 204 of the measuring device 200 in any suitable manner. Exemplary suitable manners of creating the markings include the printing of the markers, the molding of the shaft about an insert containing the markings, or the like. In one or more aspects of the present invention the indicia may be selected or printed such that it is visible in low light conditions. The indicia may be radiopaque in some aspects of the present invention.

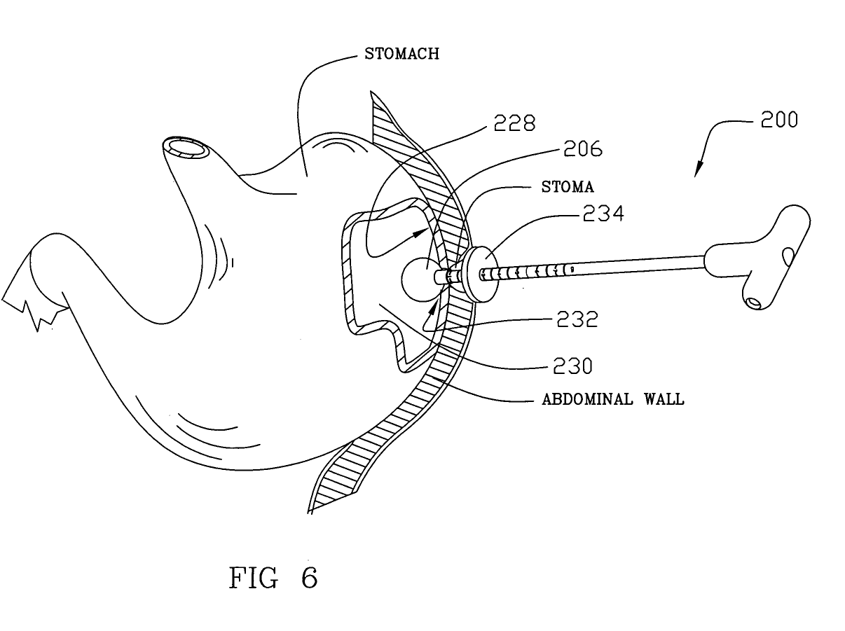

It will be appreciated by those having skill in the art that the markings 222 will desirably be indicative of the distance from the proximalmost point at which the sleeve 206 contacts the inner surface 228 of an inner body cavity 230 within the patient to which the tract extends when the measuring device 200 is properly positioned within the patient and the sleeve 206 and expandable cavity 220 are expanded or deployed (e.g. when the expandable sleeve 206 or cavity 220 is fully expanded or deployed or when the expandable sleeve 206 or cavity 220 may be less than fully expanded or deployed yet provides sufficient resistance to displacement). A discussion of the desired positioning of the measuring device at the time of measurement is described in more detail below. However, for purposes of understanding the proximalmost point at which the sleeve 206 contacts or rests against the inner surface 228 of the inner body cavity 230 within the patient to which the tract to be measured extends when the measuring device is properly positioned within the patient is exemplarily illustrated at point 232 in FIG. 6.

The head 202 of the measuring device of the present invention may be attached to the proximal end of the shaft 204 in any suitable manner. Exemplary ways of attaching the head 202 to the shaft 204 include but are not limited to adhesive securement or overmolding.

The measuring device 200 of the present invention may further include a valve 207 to regulate fluid flow into or out of the second lumen 214 and/or expandable cavity 220 and thereby control expansion or deployment of expandable cavity. Such a valve 207 will desirably be located in the opening of the head 202. Any suitable valve is contemplated. An exemplary valve may be a luer lock inflation valve such as that found in the MIC-KEY* low profile gastrostomy feeding tube (available from Ballard Medical Products, a wholly owned subsidiary of the assignee of the present invention).

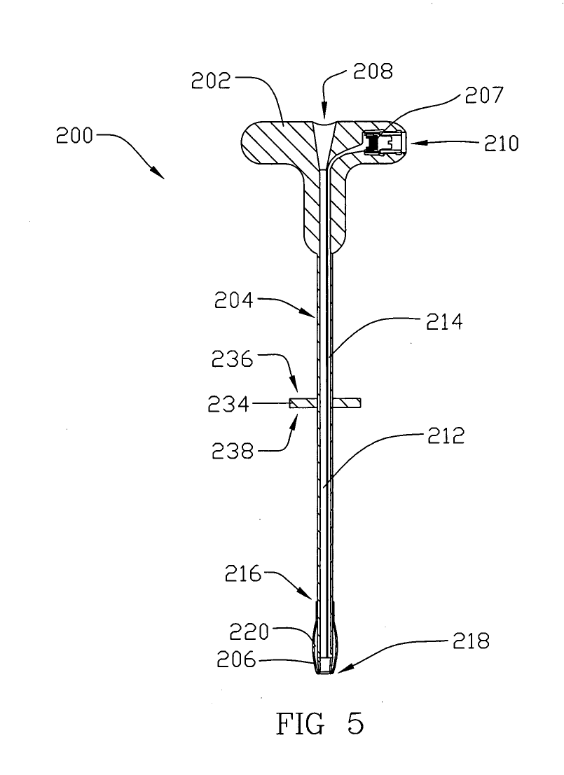

Although the sleeve 206 is shown in FIGS. 3-4 as being attached to the shaft 204 such that proximal and distal cuffs 224, 226, respectively, are formed about the shaft and generally extend away from the expandable cavity 220 created between the cuffs 224, 226, it is contemplated that one or more of the sleeve ends may be attached to the shaft 204 in an inverted or folded under fashion such that the resulting cuff or point of attachment extends inward relative to the expandable cavity 220, such as suggested in FIG. 5. Such attachments are described in more detail in the context of enteral feeding catheters in U.S. patent application Ser. No. 10/307,057 which is assigned to the assignee of the present invention, and which is incorporated in its entirety herein for all purposes.

It will be appreciated that the sleeve, balloon, etc. may be formed by any acceptable process, including for example, injection molding, dipping, compression molding, extrusion, or the like. Furthermore, it is contemplated that the sleeve 206 may be secured to the shaft 204 of the measuring device 200 in any suitable manner, including, for example, by adhesive or overmolding.

It will also be appreciated that the sleeve may be formed so as to allow for controlled expansion or deployment in a particular direction or limit expansion in another. Alternately, a sleeve may be designed to assume suitable shapes other than the traditional rounded shape. The sleeve may be designed so as upon expansion it forms such exemplary shapes as tire shaped, apple shaped, oblong, or the like. It will be appreciated that the ability of a stoma or tract measuring device to include a sleeve which is sized, configured, and attached to the shaft in the same or similar fashion to that which is included on the catheter to be placed within the patient will allow a more accurate sizing in some instances. That is, for example, if the enteral feeding catheter to be placed has a sleeve or balloon shaped differently than that on the tract measuring device, it is possible that upon placement of the enteral feeding catheter that the catheter could fit the patient tighter or looser than desired. In some instances the deviation between the shapes or manner of attachment of the sleeve or balloon on the shaft of the measuring device and enteral feeding catheter may provide negligible or inconsequential differences in the measurements taken, although in some instances the resulting measurements may vary significantly. It will be appreciated that the greater the deviation the more likely a significantly difference in measurements is to occur.

Having generally described an aspect of a measuring device of the present invention, it will be appreciated that the first lumen 212 is adapted to slidably receive a guidewire (not shown) therethrough. That is, unlike prior tract measuring devices, the measuring device 200 of the present invention has a lumen 212 that extends along the length of the measuring device 200 and is open at both ends. Previous devices did not provide for the passage of a guidewire or the like therethrough, but were rather only intended to measure the length of the stoma. The present invention is advantageous over those prior devices especially where a guidewire is to be used for the placement of the catheter the measurement is being taken for. That is, with those enteral catheters which are longer or extend further into a patient than gastrostomy catheters generally do, there is frequently a need and/or desire to use a guidewire to enable or facilitate placement of the catheter. In most cases the guidewire is placed through an existing catheter prior to removal of the existing catheter, the thought being that the guidewire will allow the replacement catheter to slide over the guidewire into the desired position within the patient. However, previous measuring devices which did not include a separate lumen which could accommodate the passage of such a guidewire required either the removal of the guidewire or that the prior measuring device be inserted into the stoma of a patient with the guidewire being forced to the side of the stoma. In those instances in which the guidewire is pushed to the side of the stoma so as to allow insertion of the measuring device, it is common for the guidewire or at least the distal end thereof to become displaced upon insertion or removal of the measuring device and/or upon expansion of a retention mechanism. In any case such premature dislocation or displacement of the guidewire is undesirable and generally requires repositioning of the guidewire. The repositioning can necessitate an additional invasive procedure. The present invention overcomes this obstacle and provides for accurate tract length determinations.

Another aspect of the present invention is directed to a dual lumen tract measuring device. The dual lumen measuring device includes a shaft and a retention member. The shaft has two lumens, a length, and scale indicia along at least a portion of the length. The retention member is adapted for deployment such that once the measuring device is properly positioned, the deployed retention member may act to retain the measuring device in position while the measurement is taken. The first lumen of the dual lumen measuring device first lumen extends the length of the shaft. The second lumen is in communication with the retention member such that the retention member is deployable through communication with the second lumen. Any suitable manner of deploying the retention member is contemplated and may depend in part on the retention member included in a particular tract measuring device. Exemplary deployment possibilities include the passage or injection of a fluid through the second lumen to trigger expansion or deployment; the insertion of a wire or the like which is adapted to release or otherwise trigger the deployment of the retention member; and the release of tension on a wire or the like associated with the retention member such that upon release of the tension on the wire the retention member deploys and upon the application of sufficient tension on the wire the retention member collapses or returns to a non-deployed state.

The present invention is also directed to a method of measuring a tract within a patient. The method generally includes the steps of providing a multi-lumen measuring device such as one or more of those described above; inserting the distal end of the measuring device into the tract in the patient; deploying the sleeve of the device; positioning the measuring device such that a proximal edge of the deployed sleeve rests against an inner surface of an inner body cavity within the patient; and determining the distance between the proximal edge of the deployed sleeve and the outer surface of the patient's abdominal wall. It will be appreciated that the inner surface of an inner body cavity within the patient upon which the proximal edge of the deployed sleeve desirably rests is desirably adjacent the distal opening of the tract or stoma being measured.

It will be appreciated that the measuring device may further include scale indicia along at least a portion of the shaft, and that the step of determining the distance between the proximal edge of the expanded sleeve and the outer surface of the patient's abdominal wall includes utilizing the scale indicia. Another aspect of the present invention may include a positioning member 234 having a distal side 236 and proximal side 238 as illustrated in FIGS. 3-5. Where the tract measuring device includes a head the positioning member will be positioned between the head of the device and the sleeve. In those aspects including a positioning bar or member, the method of measuring a tract within a patient may further include moving the positioning member along the shaft of the device until the distal edge of the positioning member rests against the patient's abdominal wall. A reading or other determination of the tract length may then be determined with the assistance of the positioning member. Of course it will be appreciated that the measurement is to be taken once the tract measuring device is properly positioned.

As suggested above, the method may also include the steps of providing a guidewire, and passing the guidewire through the first opening and lumen of the measuring device. Such a step will generally be utilized before removing the existing catheter and replacing it with another catheter. Alternatively, the method may include the steps of providing a guidewire, and passing the first lumen of the measuring device over the guidewire. This step will generally be utilized where a guidewire is already positioned within a patient and it is desirous to utilize the guidewire to facilitate placement of the catheter.

Another aspect of the present invention is directed to a method of providing a system for determining the size of a tract in a patient. The method generally includes the steps of providing a tract measuring device having at least one measuring means, and providing directions for positioning the measuring device relative to the patient so as to allow a clinician to determine the size of the tract utilizing the at least one measuring means. More specifically, the step of providing a tract measuring device includes providing a measuring device having a head, a shaft, and a sleeve. The head should have at least two openings. The openings are desirably configured such that, depending on the particular embodiment, fluid or other objects may be passed therethrough. The shaft extends from the head, and the shaft has a first and second lumen disposed in communication with the two openings of the head. The first lumen of the shaft being configured for communication with a body cavity. The sleeve has a proximal end and a distal end, each end of the sleeve being attached to the shaft so as to form an expandable cavity between the sleeve and the shaft, the expandable cavity being in communication, and desirably in fluid communication with the second lumen in the shaft. As suggested herein, the measuring means can be a positioning member, a graduated or scaled indicia, or the like.

It will be appreciated that the measuring device may further include scale indicia along at least a portion of the shaft, and that the step of determining the distance between the proximal edge of the expanded sleeve and the outer surface of the patient's abdominal wall includes utilizing the scale indicia. The measuring means can be a positioning member, a graduated or scaled indicia, or the like.

As above, the first lumen of the shaft is adapted to slidably receive a guidewire therethrough.

The present invention is also directed to a method of providing a system for determining the size of a tract in a patient. The method generally includes the steps of providing a measuring device having at least one measuring means, such as those described above; and providing directions for positioning the measuring device relative to the patient so as to allow a clinician to determine the size of the tract utilizing the at least one measuring means. The directions should generally provide for or describe the steps of: inserting the distal end of the measuring device into the tract in the patient; expanding the sleeve of the device, positioning the measuring device such that the proximal edge of the expanded or deployed sleeve rests against an inner surface of an inner body cavity within the patient, and determining the distance between the proximal edge of the expanded sleeve and the outer surface of the patient's abdominal wall.

As discussed above, such a measuring device may further include scale indicia along at least a portion of the shaft. Accordingly, with those aspects of the present invention including the scale indicia, the step of determining the distance between the proximal edge of the expanded sleeve and the outer surface of the patient's abdominal wall may include utilizing the scale indicia.

As above, measuring devices of the present invention may further include a positioning member having a distal side and proximal side, and accordingly, the method of determining the size of a tract within a patient may further include moving the positioning bar along the shaft of the device until the distal edge of the positioning member rests against the patient's abdominal wall when the measuring device is properly positioned within the patient.

While the invention has been described in detail with respect to specific aspects thereof, those skilled in the art, upon obtaining an understanding of the invention, may readily conceive of alterations to, variations of, and equivalents to the described aspects and the processes for making them. The invention may be embodied in other specific forms without departing from the scope and spirit of the inventive characteristics thereof. The present aspects therefore are to be considered in all respects as illustrative and not restrictive, the scope of the invention being indicated by the appended claims rather than by the foregoing description, and all changes which come within the meaning and range of equivalency of the claims are therefore intended to be embraced therein.

We claim:

1. A multi-lumen guidewire accessible tract measuring device comprising:
a head having at least two openings;
a shaft extending from the head, the shaft having a first and second lumen disposed in communication with the at least two openings, the first lumen configured for communication with a body cavity; and
a sleeve having a proximal end and a distal end, wherein each end of the sleeve is attached to the shaft;
wherein an expandable cavity is formed between the sleeve and the shaft and is in communication with the second lumen in the shaft.

2. The measuring device of claim 1 further comprising scale indicia along at least a portion of the shaft.

3. The measuring device of claim 1 further comprising a valve to regulate fluid flow into or out of the expandable cavity and/or second lumen.

4. The measuring device of claim 1, wherein the first lumen is adapted to slidably receive a guidewire therethrough.

5. A dual lumen tract measuring device comprising:
a shaft having two lumens, a length, and scale indicia along at least a portion of the length; and
a retention member, the retention member being adapted for deployment;
wherein the first lumen extends the length of the shaft, the second lumen being in communication with the retention member such that the retention member is deployable through communication with the second lumen.

6. A method of measuring a tract in a patient comprising:
providing a measuring device of claim 1;
inserting the distal end of the measuring device into the tract in the patient;
expanding the sleeve of the device;
positioning the measuring device such that a proximal edge of the expanded sleeve rests against an inner surface of an inner body cavity within the patient; and
determining the distance between the proximal edge of the expanded sleeve and the outer surface of the patient's abdominal wall.

7. The method of claim 6, the measuring device further comprising scale indicia along at least a portion of the shaft, and wherein the step of determining the distance between the proximal edge of the expanded sleeve and the outer surface of the patient's abdominal wall includes utilizing the scale indicia.

8. The method of claim 6, the measuring device further comprising a positioning member having a distal side and proximal side, and wherein the method further includes moving the positioning bar along the shaft of the device until the distal edge of the positioning member rests against the patient's abdominal wall.

9. The method of claim 6 further comprising the steps of providing a guidewire, and passing a guidewire through the first opening and lumen of the measuring device.

10. The method of claim 6 further comprising the steps of providing a guidewire, and passing the first lumen of the measuring device over the guidewire.

11. A method of providing a system for determining the size of tract in a patient, comprising the steps of:
providing a tract measuring device having at least one measuring means, the measuring device comprising:
a head having at least two openings;
a shaft extending from the head, the shaft having a first and second lumen disposed in communication with the at least two openings, the first lumen configured for communication with a body cavity; and
a sleeve having a proximal end and a distal end, wherein each end of the sleeve is attached to the shaft;
wherein an expandable cavity is formed between the sleeve and the shaft and is in communication with the second lumen in the shaft; and
providing directions for positioning the measuring device relative to the patient so as to allow a clinician to determine the size of the tract utilizing the at least one measuring means,

12. The method of claim 11, wherein the measuring device further comprises scale indicia along at least a portion of the shaft, and the method further comprises the step of utilizing the scale indicia to determine the size of the tract.

13. The method of claim 11, wherein the first lumen is adapted to slidably receive a guidewire therethrough.

14. A method of providing a system for determining the size of a tract in a patient, comprising the steps of:
providing a measuring device of claim 1; and
providing directions for positioning the measuring device relative to the patient so as to allow a clinician to determine the size of the tract, the directions
inserting the distal end of the measuring device into the tract in the patient;
expanding the sleeve of the device;
positioning the measuring device such that a proximal edge of the expanded sleeve rests against an inner surface of an inner body cavity within the patient; and
determining the distance between the proximal edge of the expanded sleeve and the outer surface of the patient's abdominal wall.

15. The method of claim 14, the measuring device further comprising scale indicia along at least a portion of the shaft, and wherein the step of determining the distance between the proximal edge of the expanded sleeve and the outer surface of the patient's abdominal wall includes utilizing the scale indicia.

16. The method of claim 14, the measuring device further comprising a positioning member having a distal side and proximal side, and wherein the method further includes moving the positioning bar along the shaft of the device until the distal edge of the positioning member rests against the patient's abdominal wall.

17. The method of claim 14 further comprising the steps of providing a guidewire, and passing a guidewire through the first opening and lumen of the measuring device.

18. The method of claim 14 further comprising the steps of providing a guidewire, and passing the first lumen of the measuring device over the guidewire.
